(19) United States
(12) Patent Application Publication
(10) Pub. No.: US 2015/0246239 A1
(43) Pub. Date: Sep. 3, 2015

(54) HEALTH CARE METHOD INCLUDING LIGHTING THERAPY, ART THERAPY, MUSIC THERAPY, AND COGNITIVE THERAPY

(71) Applicant: UNIVERSITY INDUSTRY COOPERATION GROUP OF KYUNG-HEE UNIVERSITY ET AL, Dongdaemun-gu Seoul (KR)

(72) Inventors: Oh Byung Kwon, Seongnam-si (KR); Jae Moon Sim, Daegu (KR)

(73) Assignee: UNIVERSITY-INDUSTRY COOPERATION GROUP OF KYUNG-HEE UNIVERSITY, Yongin-si, Gyeonggi-do (KR)

(21) Appl. No.: 14/433,505

(22) PCT Filed: Oct. 4, 2013
(86) PCT No.: PCT/KR2013/008908

§ 371 Date: Apr. 3, 2015

(30) Foreign Application Priority Data
Oct. 5, 2012 (KR) 10-2012-0110710
Oct. 26, 2012 (KR) 10-2012-0119849

(51) Int. Cl. A61N 5/06

(52) U.S. Cl. CPC A61N 5/06; A61N 5/0618; G06Q 50/24

(57) ABSTRACT

The present invention relates to a health care method using a light therapy device and an auxiliary therapy device, the health care method can relax the mind and body of a user by controlling the light therapy device and the auxiliary therapy device on the basis of a user's condition or changes in the user's condition, and accessorily, the mental state of the user.

### TECHNICAL FIELD

The present invention relates to a health management method using a lighting therapeutic device and an auxiliary therapeutic device, and more particularly, to such a health management method which can relax a user's mind and body through the control of a lighting therapeutic device and an auxiliary therapeutic device based on a current user's condition or a change in the user's condition, and auxiliarily the user's psychological state.

### BACKGROUND ART

Entry into advanced countries further increases an interest in health. Thus, a health care service is one of the promising industries. In particular, wellness meaning health and happiness began to be spotlighted as a core value of a new industrial field including medical wellness tourism and the like. In addition, a program for promoting wellness is continuously introduced into employees, and thus management of wellness is becoming increasingly important in the improvement of individual or organizational performance in enterprises

Furthermore, the importance of health care is emphasized as a preventive medicine service, rather than a therapeutic service that medical treatment is traditionally performed on an affected part of a patient's body. In addition, the importance of healthcare is increasing in the psychological therapy field as well as the physical therapy field. Herein, a therapeutic method in the psychological therapy field can employ various methods including a music therapy, an art therapy, a lighting therapy, and cognitive therapy as well as a traditional mental counseling.

However, the conventional psychological therapy method entails a problem in that since a patient must reserve the clinic time and visit a hospital in the reserved clinic time to undergo a medical examination, and use a predesigned limited therapeutic program, it is impossible to perform a psychological therapy reflecting the characteristics of individuals. If a ubiquitous technology can be applied to the psychological therapy field, the user can continuously receive an appropriate psychological therapy service without any restriction of place and time when a psychological therapy is needed, thereby increasing the psychological therapy effect.

### DISCLOSURE OF INVENTION

### Technical Problem

Accordingly, the present invention has been made to solve the above-mentioned problems occurring in the conventional psychological therapy service, and it is an object of the present invention to provide a health management method which can provide a lighting therapy to a user in real-time depending on a user's condition and a user's psychological state without any restriction of space and time.

Another object of the present invention is to provide a health management method which can provide a lighting therapy suitable for a change in the user's condition in real-time based on a change between a previous user's condition and a current user's condition.

Still another object of the present invention is to provide a health management method which can primarily determine a primary therapeutic method mapped to a user's condition based on the user's condition, and determine a final therapeutic method mapped to the user's psychological state to provide the determined final therapeutic method to the user.

Yet another object of the present invention is to provide a health management method which simultaneously provides a music therapy, an art therapy, and an acupressure therapy centering on a lighting therapy depending on the user's condition and the user's psychological state so that the user's mind and body can be effectively cured through various therapeutic methods.

### Technical Solution

To achieve the above object, in accordance with one embodiment of the present invention, there is provided a health management method including: collecting user's location information, user information, environmental information and information regarding the operational state of a user peripheral device; determining a user's condition based on the user's location information or the information regarding the operational state of the user peripheral device; determining a user's psychological state mapped to the user information and the environmental information in the user's condition based on the user information and the environmental information; determining a primary lighting therapy based on the user's condition, and determining a final lighting therapy mapped to the user's psychological state in the determined primary lighting therapy based on the user's condition; and retrieving a main lighting and an auxiliary lighting of a lighting therapeutic device disposed in a space where the user is located based on the user's location information, and controlling the operation and illuminance of the main lighting and the auxiliary lighting of the lighting therapy unit based on the determined final lighting therapy.

Preferably, the health management method in accordance with the present invention may further include: determining whether or not there exists an auxiliary therapy mapped to the user's condition or the user's psychological state based on the user's condition or the user's psychological state; determining, if there exists the auxiliary therapy, whether or not the auxiliary therapeutic device to perform the auxiliary therapy exists in the space where the user is located based on the user's location information; and controlling the operation of the auxiliary therapeutic device based on the determined auxiliary therapy if the auxiliary therapeutic device to perform the auxiliary therapy exists.

In the health management method in accordance with the present invention, the step of determining the user's condition may include determining the user's condition at a previous unit time based on the user's location information or the information regarding the operational state of the user peripheral device at the previous unit time and determining the user's condition at a current unit time based on the user's location information or the information regarding the operational state of the user peripheral device at the current unit time. In addition, the step of determining the lighting therapy may include determining a primary lighting therapy mapped to a change between the user's condition at the previous unit time and the user's condition at the current unit time.

In the health management method in accordance with the present invention, the main lighting of the lighting therapeutic device may control an illuminance of the space where the user is located, and the auxiliary lighting of the lighting therapeutic device may control a color temperature or color of the space where the user is located.

In the health management method in accordance with the present invention, the auxiliary therapeutic device may include: a music therapeutic unit for playing back music contents mapped to the user's condition or the user's psychological state; an art therapeutic unit for displaying art contents mapped to the user's condition or the user's psychological state to the user; and an acupressure therapeutic unit for executing a acupressure program mapped to an acupressure program mapped to the user's condition or the user's psychological state.

In the health management method in accordance with the present invention, the user information may include user profile information including the user's age, sex, occupation, and hobbies, and user's body information including the user's weight, blood pressure, body temperature, physical activity level, stress index, fatigue index, and discomfort index. In addition, the environmental information may include current temperature, humidity, illuminance, noise, weather, date, and season.

To achieve the above object, in accordance with another embodiment of the present invention, there is provided a health management device including: a collection unit for collecting user's location information, user information, environmental information and information regarding the operational state of a user peripheral device; a user's condition determination unit for determining a user's condition based on the user's location information or the information regarding the operational state of the user peripheral device; a psychological index determination unit for determining a user's psychological state mapped to the user information and the environmental information in the user's condition based on the user information and the environmental information; a therapeutic method determination unit for determining a primary lighting therapeutic method based on the user's condition, and determining a final lighting therapeutic method mapped to the user's psychological state in the determined primary lighting therapeutic method based on the user's condition; and a therapy control unit for retrieving a lighting therapeutic device or an auxiliary therapeutic device disposed in a space where the user is located based on the user's location information, and controlling the operation of the lighting therapeutic device or the auxiliary therapeutic device based on the determined final lighting therapy.

In the health management device in accordance with another embodiment of the present invention, the user's location information may be motion detection information received from a motion detection sensor disposed in the space where the user is located or location information received from a user terminal held by the user.

In the meantime, the collection unit may receive information regarding the operational state of the user peripheral device, which indicates the current operational state of the user peripheral device based on the operational state of the user peripheral device, which is transmitted thereto from the user peripheral device.

In the health management device in accordance with another embodiment of the present invention, the lighting therapeutic device may include: a main lighting for controlling an illuminance of the space where the user is located; and an auxiliary lighting for controlling a color temperature or color of the space where the user is located.

Preferably, the auxiliary therapeutic device may include: a music therapeutic unit for playing back music contents mapped to the user's condition or the user's psychological state; an art therapeutic unit for displaying art contents mapped to the user's condition or the user's psychological state to the user; and an acupressure therapeutic unit for executing a acupressure program mapped to an acupressure program mapped to the user's condition or the user's psychological state.

Preferably, the health management device in accordance with the present invention may further include a adjustment setting unit for re-adjusting the determined final therapeutic method or setting and storing the re-adjusted final therapeutic method.

### Advantageous Effects

The health management method according to the embodiment of the present invention as constructed above have the following various advantageous effects over the conventional health management method.

First, the health management method in accordance with the present invention provides a lighting therapy, a music therapy, an art therapy, and an acupressure therapy, which are suitable for the user's condition and the user's psychological state, in the space where the user is located so that the therapeutic effect of the user's mind and body can be maximized without any restriction of space and time.

Second, the health management method in accordance with the present invention provides a lighting therapy to the user by reflecting a change between a previous user's condition and a current user's condition so that the users' condition can be accurately determined depending on a change in the user's condition and a health management method suitable for the user's condition can be provided.

Third, the health management method in accordance with the present invention primarily determines a primary therapeutic method mapped to a user's condition based on the user's condition, and again determines a final therapeutic method mapped to the user's psychological state in the primary therapeutic method so that a health management method optimized for the current state of the user's mind and body can be provided.

Fourth, the health management method in accordance with the present invention simultaneously provides a music therapy, an art therapy, and an acupressure therapy centering on a lighting therapy depending on the user's condition and the user's psychological state so that the user's mind and body can be effectively cured through a combination of various therapeutic methods.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a functional block diagram illustrating a health management system in accordance with the present invention;

FIG. 2 is a functional block diagram illustrating a health management device in accordance with the present invention;

FIG. 3 is a functional block diagram illustrating a therapeutic method determination unit in accordance with the present invention;

FIG. 4 is a functional block diagram illustrating a therapy control unit in accordance with the present invention;

FIG. 5 is a flow chart illustrating a health management method in accordance with the present invention;

FIG. 6 is a flow chart more specifically illustrating a process of determining a therapeutic method in accordance with the present invention;

FIG. 7 is a table illustrating one example of a primary lighting therapeutic method stored in a therapeutic database; and

FIG. 8 is a table illustrating one example of a final lighting therapeutic method stored in a therapeutic database.

### BEST MODE FOR CARRYING OUT THE INVENTION

Now, a health management method in accordance with an embodiment of the present invention will be described hereinafter in detail with reference to the accompanying drawings.

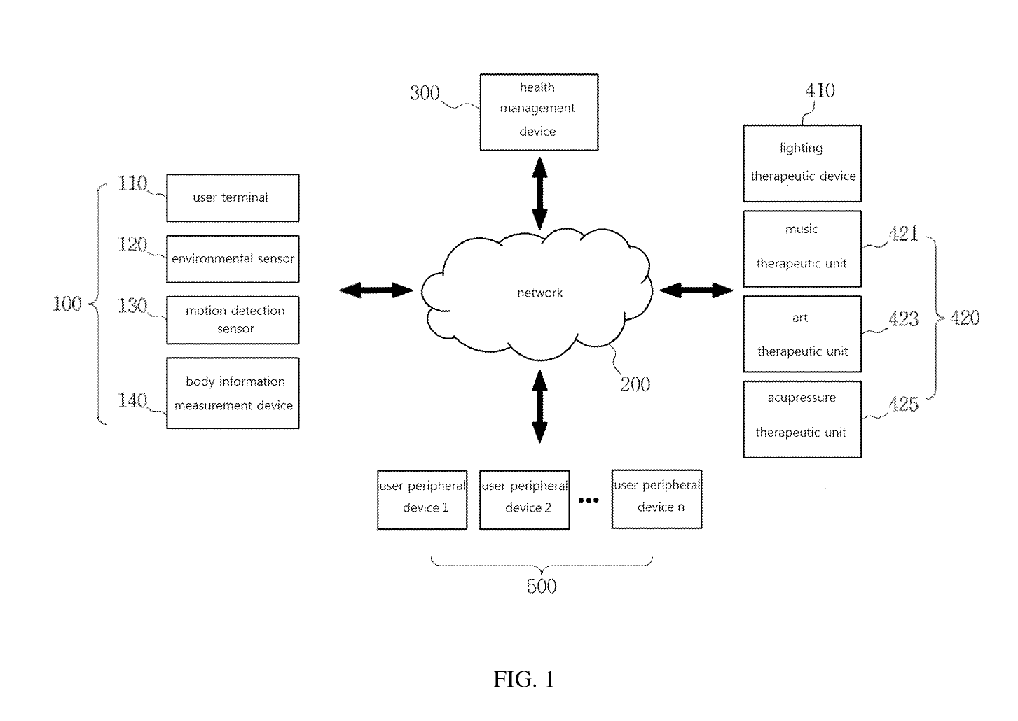

FIG. 1 is a functional block diagram illustrating a health management system in accordance with the present invention.

Referring to FIG. 1, more specifically, a wired or wireless network 200 is connected to a plurality of information collection devices 100, a plurality of user peripheral devices 500 disposed in a user space, a health management device 300 for determining a therapeutic program for treating a user's mind and body based on information collected by the information collection devices 100 and the user peripheral devices 500, and a plurality of therapeutic units 400 for executing a therapeutic program determined by the health management device 300 under the control of the health management device 300.

The information collection device 100 will be described hereinafter in more detail.

The information collection device 100 serves to collect information on a place where a user is currently located, user information, and environmental information. The information collection device 100 that can be used in the present invention includes a user terminal 110, an environmental sensor 120, a motion detection sensor 130, and a body information measurement device 140. The information collection device 100 may include a variety of kinds of devices depending on a field to which the present invention is applied, besides the user terminal 110, the environmental sensor 120, the motion detection sensor 130, and the body information measurement device 140, which are fall within the scope of the present invention.

Herein, the user information conceptually includes user profile information and user's body information. The user profile information is user personal information such as a user's age, name, sex, weight, occupation, hobbies and the like. The user's body information is information associated with the user's body, such as the user's body temperature, weight, blood pressure, physical activity level, stress index, fatigue index, discomfort index and the like. On the other hand, environmental information is information indicating the environmental state of a space where the user is located, such as current temperature, humidity, illuminance, noise, weather, date, and season and the like.

The user profile information is collected by the user terminal 110. The user inputs his or her name, sex, age, occupation, hobbies and the like personally through the user terminal 110. The inputted user profile information is transmitted to the health management device 300 through the network 200. Meanwhile, the user's body information is collected by body information measurement device 140. The body information measurement device 140 measures the user's various body information, for example, such as the user's body temperature, weight, blood pressure, physical activity level and the like. The measured body information of the user is transmitted to the health management device 300 through the network 200. On the other hand, the user's stress index, fatigue index, and discomfort index are measured in the user's response to a question provided to the user by the user terminal 110 and is transmitted to the health management device 300 through the network 200.

As one example, the user terminal 110 is used to determine the user's location information. The user terminal 110 is mounted with a device for determining the user's position, e.g., an RFID chip and a PGS module. In the case where the user moves with him or her carrying it, the user's location information is transmitted to the health management device 300 through the network 200. As another example, the motion detection sensor 130 is disposed in a user space so as to determine the user's location information. In the case where the user is located in the user space, the user's location information is acquired by the motion detection sensor 130, and is transmitted to the health management device 300 through the network 200.

On the other hand, the user peripheral device 500 is disposed in the user space, and means a device such as a television set, an air-conditioner, a desk lamp, a music playback device and the like, which are used by the user. The user peripheral device 500 provides information regarding whether or not the devices are in an ON/OFF state and information regarding the functional and operational state of the devices to the health management device 300 through the network 200.

The health management device 300 determines the user's condition or psychological state based on the user's location information, the user information, the environmental information, and the information regarding the operational state of the user peripheral devices 500, which are transmitted thereto from the information collection devices 100 and the user peripheral devices 500, and selects lighting therapy or auxiliary therapy based on the determined user's condition or psychological state. The auxiliary therapy is a medical treatment that can complement the lighting therapy depending on the user's condition or psychological state. Art therapy, music therapy, acupressure therapy and the like can be used as the auxiliary therapy. A variety of kinds of auxiliary therapies can be used depending on a field to which the present invention is applied.

The therapeutic device 400 is a device that performs a medical treatment depending on a therapeutic program determined by the health management device 300, and includes a lighting therapeutic device 410 for performing the lighting therapy and an auxiliary therapeutic device 420.

The lighting therapeutic device 410 treats the user's physical disability and mental disorder using light. An appropriate color temperature or color provided to the user depending on the user's psychological and emotional condition is effective in the treatment of the user's mental or emotional disorder. Such light can be expressed by color or color temperature using RGB. In particular, the color temperature is used to represent natural light and the expressed color of a light source is displayed as an absolute temperature of the light source. When the color of the light source is based on red color series, the color temperature of the light source is low whereas when the color of the light source is based on blue color series, the color temperature of the light source is high. The lighting therapeutic device 410 provides light having an appropriate color or color temperature through an artificial light source as a lighting device to alleviate the user's stress, depression and fatigue. This lighting therapy has been proven as being effective in the improvement of physical disability and emotional disorder for a long time. For example, according to the results of a study, approximately 5% of pregnant women for childbirth suffer from severe gravid depression. According to a report, there exists no drug treatment method to effectively improve gravid depression (Oren, D. A., “An open trail of morning light therapy for treatment of antepartum Depression”, The American Journal of Psychiatry, 2006). However, it has been confirmed that a depression relieving effect of about 49% was achieved by using natural light in the morning. In addition, the above study has recognized that the lighting therapy is effective in the treatment of various kinds of sleep disorders and has suggested a guideline associated with an appropriate color temperature, illuminance and exposure time for the lighting therapy according to the sleep disorders.

In the meantime, the auxiliary therapeutic device 420 includes a music therapeutic unit 421 that provides music contents to the user depending on the user's condition or psychological state, an art therapeutic unit 423 that provides art contents to the user depending on the user's condition or psychological state, and an acupressure therapeutic unit 425 that provides a acupressure service to the user depending on the user's condition and psychological state.

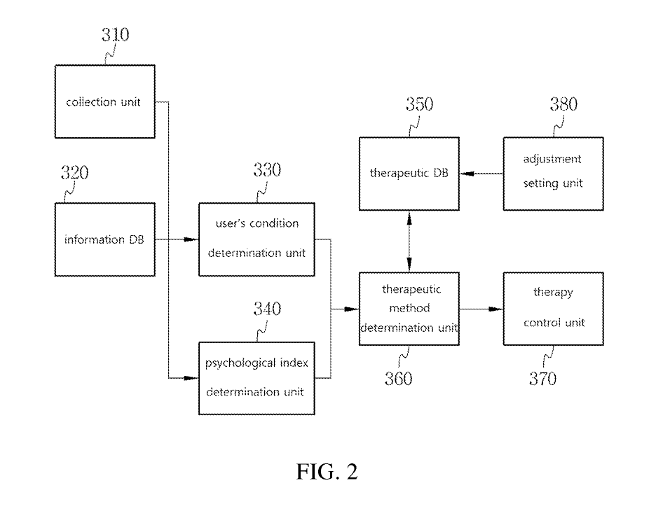

FIG. 2 is a functional block diagram illustrating a health management device in accordance with the present invention.

Referring to FIG. 2, more specifically, a collection unit 310 receives the user's location information, user information or environmental information from the information collection device 100 through the network 200 or receives information regarding the operational state of the user peripheral device 500 from the user peripheral device 500. The collection unit 310 stores and registers the received user's location information, user information or environmental information in an information database 320.

A user's condition determination unit 330 determines the user's condition in a place where the user is currently located based on the user's location information and information regarding the operational state of the user peripheral device. A psychological index determination unit 340 determines the user's psychological state based on user information and environmental information. More specifically, the user's condition determination unit 330 determines a place where the user is currently located based on the user's location information, and then determines the user's condition based on information regarding the operational state of the user peripheral device and a place where the user is currently located. Meanwhile, the psychological index determination unit 340 determines the user's psychological state that is personalized depending on the user's stress index, fatigue index, and discomfort index based on the environmental information. Herein, the stress index, the fatigue index and the discomfort index are values that indicate the relative sizes of stress, fatigue and discomfort which the user feels under the same environment, and thus the psychological index determination unit 340 determines the user's psychological state such as stress, fatigue, and discomfort based on environmental information of the place where the user is located.

A therapeutic method determination unit 360 determines a primary lighting therapeutic method mapped to the user's condition in the therapeutic database 350, and again determines a final therapeutic method mapped to the user's psychological state in the determined primary therapeutic method. A therapy control unit 370 retrieves the lighting therapeutic device 410 or the auxiliary therapeutic device 420 disposed in the space where the user is located based on the user's location information, and controls the operation of the lighting therapeutic device 410 or the auxiliary therapeutic device 420 according to the determined final therapeutic method.

Preferably, an adjustment setting unit 380 adjusts and sets the contents of the lighting therapy or the auxiliary therapy in response to a user command which is inputted for the final therapeutic method determined by the therapeutic method determination unit 360, and updates and stores the adjusted and set contents of the lighting therapy or the auxiliary therapy in the therapeutic database 350. The therapeutic database 350 initially stores the contents of the lighting therapy or the auxiliary therapy of an expert according to the user's condition or psychological state. The user who has received a therapeutic treatment of the lighting therapeutic device 410 or the auxiliary therapeutic device 420 may update the therapeutic contents of the therapeutic database 350 to the therapeutic contents which are personalized to the user and receive optimized therapeutic contents later.

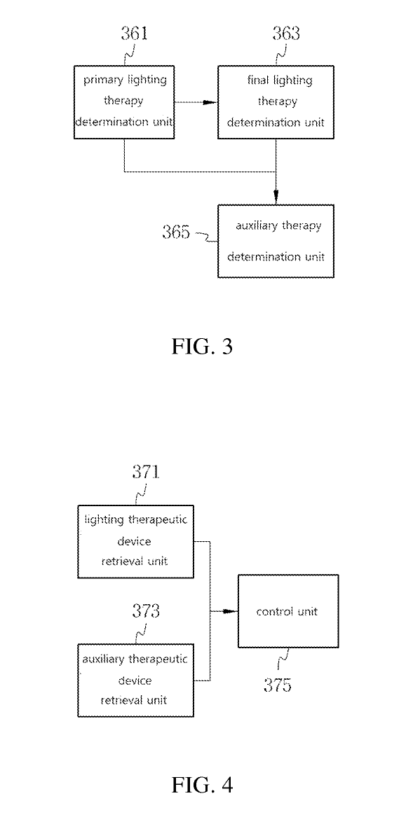

FIG. 3 is a functional block diagram illustrating a therapeutic method determination unit in accordance with the present invention.

Referring to FIG. 3, more specifically, a primary lighting therapy determination unit 361 determines a primary lighting therapy mapped to the user's condition among lighting therapies stored in the therapeutic database 350. A final lighting therapy determination unit 363 retrieves a lighting therapy mapped to the user's psychological state in the primary lighting therapy mapped to the user's condition from the therapeutic database 350, and determines the lighting therapy mapped to the user's psychological state in the primary lighting therapy as a final lighting therapy. In the present invention, the final lighting therapy determination unit 363 primarily determines the lighting therapy mapped to the user's condition and again determines the final lighting therapy based on the user's psychological state so that different lighting therapies can be provided depending on the user's psychological state which varies even under the same user condition, thereby maximizing the lighting therapeutic effect.

In the meantime, an auxiliary therapy determination unit 365 retrieves whether or not there is an auxiliary therapy for complementing the determined final lighting therapy, and determines an auxiliary therapy that complements the final lighting therapy.

FIG. 4 is a functional block diagram illustrating a therapy control unit in accordance with the present invention;

Referring to FIG. 4, more specifically, a lighting therapeutic device retrieval unit 371 retrieves a main lighting and an auxiliary lighting to perform the final lighting therapy in a place where the user is located based on the determined final lighting therapy, and an auxiliary therapeutic device retrieval unit 373 retrieves the music therapeutic unit, the art therapeutic unit or the acupressure therapeutic unit to perform the auxiliary therapy in a place where the user is located or a surrounding place. Preferably, the main lighting, the auxiliary lighting, and the auxiliary therapeutic device which are disposed in the user space are stored and registered as location information and identifiers in the information database 320. The lighting therapeutic device retrieval unit 371 or the auxiliary therapeutic device retrieval unit 373 retrieves the main lighting, the auxiliary lighting, and the auxiliary therapeutic device, which are disposed in the space where the user is currently located based on the user's location information using the identifiers.

Herein, the main lighting of the lighting therapeutic device implements illuminance of the space where the user is located, and the auxiliary lighting of the lighting therapeutic device implements the color temperature or color of the space where the user is located.

A control unit 375 controls the operation of the retrieved lighting therapeutic device or auxiliary therapeutic device based on the retrieved final lighting therapy or auxiliary therapy and provides the final lighting therapy and the auxiliary therapy to the user. Preferably, the environmental sensor re-detects illuminance, noise and the like when the main lighting, the auxiliary lighting or the auxiliary therapeutic device is implemented, and the control unit 375 controls the operation of the main lighting, the auxiliary lighting or the auxiliary therapeutic device based on the re-detected environmental information. For example, the control unit 375 controls the illuminance or color change of the main lighting and auxiliary lighting, or the volume of the music therapeutic unit so as to adjust a difference between an illuminance of the re-detected environmental information and an illuminance of the final lighting therapy or a difference between a noise of the re-detected environmental information and a noise of the auxiliary therapy.

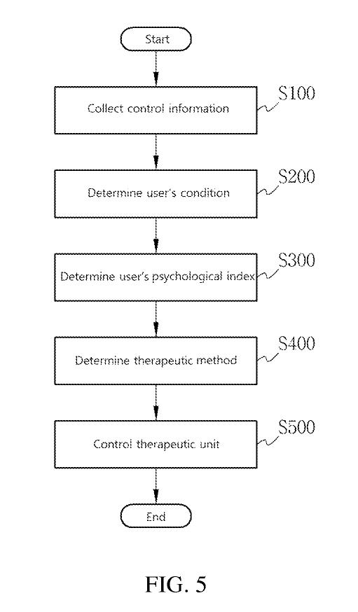

FIG. 5 is a flow chart illustrating a health management method in accordance with the present invention.

Referring to FIG. 5, more specifically, the health management device receives the user's location information, the user profile information, the user's body information, and the environmental information from the information collection device 100, or receives the registered information regarding the operational state of the user peripheral device from the user peripheral device, and stores and registers these information in the information database (S100).

The health management device determines the user's condition based on the user's location information or the information regarding the operational state of the user peripheral device (S200), and determines the user's psychological state mapped to the user information and the environmental information based on the user information and the environmental information (S300).

In case of determining the user's condition, for example, if a space where a user is currently located is a library and a desk lamp disposed on a desk among the user peripheral devices disposed in the user space of the library is operated in an ON state, the health management device determines the user's condition to be a condition in which the user is reading. On the other hand, if a space where a user is currently located is a library and a music playback device disposed on a desk among the user peripheral devices disposed in the user space of the library is operated in an ON state, the health management device determines the user's condition to be a condition in which the user takes a break.

Preferably, in the present invention, the health management device determines a change in the user's condition based on a user's condition at a previous unit time and a user's condition at a current unit time in order to more accurately the user's condition. The term “unit time” as used herein refers to a time period during which the user's condition is changed. For example, in the case where a user was studying and currently takes a break, the previous unit time means a time period during which the user was studying and the current unit time means a time period during which the user takes a break. If the space where the user is located during the previous unit time is a library and a desk lamp disposed on a desk among the user peripheral devices disposed in the user space of the library is operated in an ON state, the health management device determines the user was studying. On the other hand, if the space where the user is located during the current unit time is a library and a music playback device disposed on a desk among the user peripheral devices disposed in the user space of the library is operated in an ON state, the health management device determines that the user takes a break. Then, the health management device determines that the user was studying and currently takes a break based on a change in the user's condition between the previous unit time and the current unit time.

In case of determining the user's psychological state, for example, if the user is a male adolescent, the fatigue index and the stress index of the study are medium and the discomfort index of the temperature is high at a current temperature of 26, the health management device determines that stress and fatigue indexes of the user's psychological state is medium, and the discomfort index is high based on the user information such as the user's age, sex, stress index, fatigue index, discomfort index and the like and the environmental information such as temperature, humidity and the like.

The health management device determines a lighting therapeutic method and an auxiliary therapeutic method based on the determined user's condition or psychological state (S400). The health management device retrieves a lighting therapeutic device or an auxiliary therapeutic device to perform the lighting therapeutic method or the auxiliary therapeutic method based on the user's location information and the identifier of the lighting therapeutic device or the auxiliary therapeutic device, and controls the performance of a lighting therapy or auxiliary therapy that is determined through the retrieved lighting therapeutic device or auxiliary therapeutic device (S500).

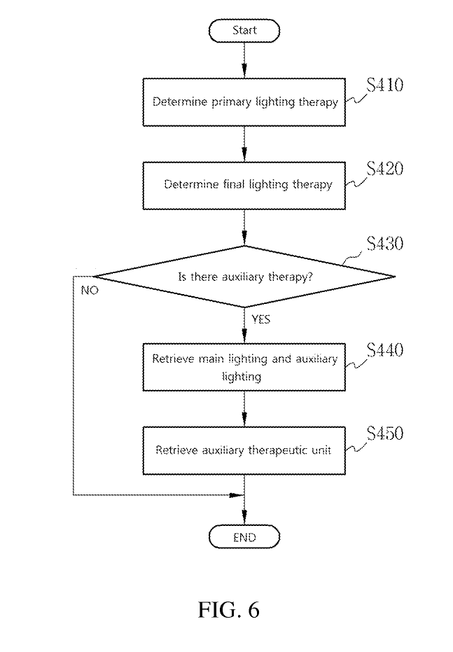

FIG. 6 is a flow chart more specifically illustrating a process of determining a therapeutic method.

Referring to FIG. 6, more specifically, the health management device determines a primary lighting therapeutic method mapped to the user's condition based on the user's condition (S410), and again determines a final lighting therapeutic method mapped to the user's psychological state based on the user's psychological state (S420).

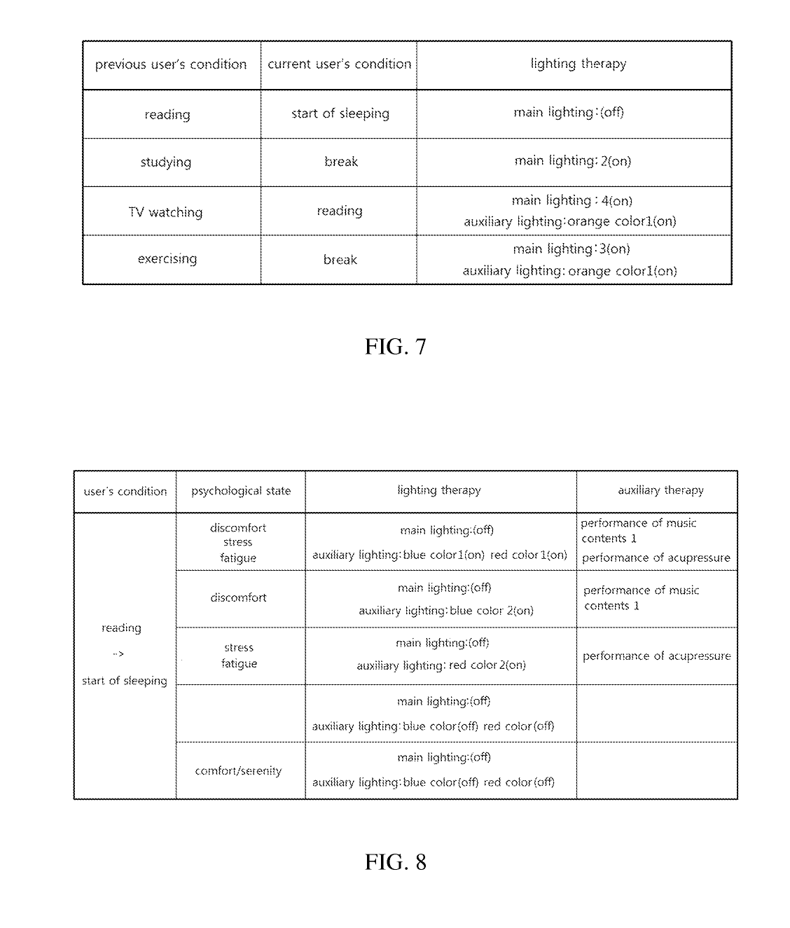

FIG. 7 is a table illustrating one example of a primary lighting therapeutic method stored in a therapeutic database, and FIG. 8 is a table illustrating one example of a final lighting therapeutic method stored in a therapeutic database.

Referring to FIGS. 7 and 8, in the case where the user was studying during the previous unit time and gest to sleep in the bedroom during the current unit time, the health management device determines a primary lighting therapeutic method mapped to the user's condition of the current unit time and the user's condition of the previous unit time. On the other hand, the primary lighting therapeutic method is determined, and then the health management device finally determines a final lighting therapeutic method according to the user's psychological state based on a discomfort index at a temperature of 26° C. and a humidity of 30% and a stress index and a fatigue index that the user feels for the study.

Meanwhile, in the case where the final lighting therapy mapped to the user's condition and the user's psychological state, the health management device determines whether or not there is an auxiliary therapeutic method to complement the determined final lighting therapy (S430). If it is determined that there is the auxiliary therapeutic method, the health management device retrieves a main lighting and an auxiliary lighting that are disposed in the space where the user is located based on the user's location information (S440), and retrieves an auxiliary therapeutic device disposed in a space where the user is located or a surrounding space based on the user's location information (S450). The therapy control unit controls the operation of the retrieved lighting therapeutic device and auxiliary therapeutic device according to the determined final lighting therapeutic method and auxiliary therapeutic method to perform the lighting therapy and the auxiliary therapy.

Meanwhile, the embodiments of the present invention as described above can be constructed by a program that can be executed in a computer and can be implemented in a universal digital computer that operates the program using a computer-readable recording medium.

The computer-readable recording medium includes a storage medium such as a magnetic storage medium (e.g., ROM, floppy disk, hard disk, or the like), an optical reading medium (e.g., CD-ROM, DVD, or the like), and a carrier wave (e.g., transfer through the Internet).

While the present invention has been described in connection with the exemplary embodiments illustrated in the drawings, they are merely illustrative and the invention is not limited to these embodiments. It will be appreciated by a person having an ordinary skill in the art that various equivalent modifications and variations of the embodiments can be made without departing from the spirit and scope of the present invention. Therefore, the true technical scope of the present invention should be defined by the technical sprit of the appended claims.

1. A health management method comprising:
collecting user's location information, user information, environmental information and information regarding the operational state of a user peripheral device;
determining a user's condition based on the user's location information or the information regarding the operational state of the user peripheral device;
determining a user's psychological state mapped to the user information and the environmental information in the user's condition based on
the user information and the environmental information;
determining a primary lighting therapy based on the user's condition, and determining a final lighting therapy mapped to the user's psychological state in the determined primary lighting therapy based on the user's condition; and
retrieving a main lighting and an auxiliary lighting of a lighting therapeutic device disposed in a space where the user is located based on the user's location information, and controlling the operation and illuminance of the main lighting and the auxiliary lighting of the lighting therapy unit based on the determined final lighting therapy.

2. The health management method according to claim 1, further comprising:
determining whether or not there exists an auxiliary therapy mapped to the user's condition or the user's psychological state based on the user's condition or the user's psychological state;
determining, if there exists the auxiliary therapy, whether or not the auxiliary therapeutic device to perform the auxiliary therapy exists in the space where the user is located based on the user's location information; and
controlling the operation of the auxiliary therapeutic device based on the determined auxiliary therapy if the auxiliary therapeutic device to perform the auxiliary therapy exists.

3. The health management method according to claim 2, wherein the step of determining the user's condition comprises determining the user's condition at a previous unit time based on the user's location information or the information regarding the operational state of the user peripheral device at the previous unit time and determining the user's condition at a current unit time based on the user's location information or the information regarding the operational state of the user peripheral device at the current unit time, and
wherein the step of determining the lighting therapy comprises determining a primary lighting therapy mapped to a change between the user's condition at the previous unit time and the user's condition at the current unit time.

4. The health management method according to claim 3, wherein the main lighting of the lighting therapeutic device controls an illuminance of the space where the user is located, and
wherein the auxiliary lighting of the lighting therapeutic device controls a color temperature or color of the space where the user is located.

5. The health management method according to claim 3, wherein the auxiliary therapeutic device comprises:
a music therapeutic unit for playing back music contents mapped to the user's condition or the user's psychological state;
an art therapeutic unit for displaying art contents mapped to the user's condition or the user's psychological state to the user; and
an acupressure therapeutic unit for executing a acupressure program mapped to an acupressure program mapped to the user's condition or the user's psychological state.

6. The health management method according to claim 1, wherein the user information comprises user profile information including the user's age, sex, occupation, and hobbies, and user's body information including the user's weight, blood pressure, body temperature, physical activity level, stress index, fatigue index, and discomfort index.

7. The health management method according to claim 1, wherein the environmental information comprises current temperature, humidity, illuminance, noise, weather, date, and season.

8. A health management device comprising:
a collection unit for collecting user's location information, user information, environmental information and information regarding the operational state of a user peripheral device;
a user's condition determination unit for determining a user's condition based on the user's location information or the information regarding the operational state of the user peripheral device;
a psychological index determination unit for determining a user's psychological state mapped to the user information and the environmental information in the user's condition based on the user information and the environmental information;
a therapeutic method determination unit for determining a primary lighting therapeutic method based on the user's condition, and determining a final lighting therapeutic method mapped to the user's psychological state in the determined primary lighting therapeutic method based on the user's condition; and
a therapy control unit for retrieving a lighting therapeutic device or an auxiliary therapeutic device disposed in a space where the user is located based on the user's location information, and controlling the operation of the lighting therapeutic device or the auxiliary therapeutic device based on the determined final lighting therapy.

9. The health management device according to claim 8, wherein the lighting therapeutic device comprises:
a main lighting for controlling an illuminance of the space where the user is located; and
an auxiliary lighting for controlling a color temperature or color of the space where the user is located.

10. The health management device according to claim 8, wherein the auxiliary therapeutic device comprises:
a music therapeutic unit for playing back music contents mapped to the user's condition or the user's psychological state;
an art therapeutic unit for displaying art contents mapped to the user's condition or the user's psychological state to the user; and
an acupressure therapeutic unit for executing a acupressure program mapped to an acupressure program mapped to the user's condition or the user's psychological state.

11. The health management device according to claim 8, further comprising a adjustment setting unit for re-adjusting the determined final therapeutic method or setting and storing the re-adjusted final therapeutic method.

12. The health management device according to claim 8, wherein the user's location information is motion detection information received from a motion detection sensor disposed in the space where the user is located or location information received from a user terminal held by the user.

13. The health management device according to claim 8, wherein the collection unit receives information regarding the operational state of the user peripheral device, which indicates the current operational state of the user peripheral device based on the operational state of the user peripheral device, which is transmitted thereto from the user peripheral device.
